(19) United States
(12) Patent Application Publication
(10) Pub. No.: US 2026/0144741 A1
(43) Pub. Date: May 28, 2026

(54) NOVEL COMPOSITIONS

(71) Applicant: DSM IP Assets B.V., Maastricht (NL)

(72) Inventor: Christine MENDROK-EDINGER, Kaiseraugst (CH)

(21) Appl. No.: 19/122,127

(22) PCT Filed: Oct. 27, 2023
(86) PCT No.: PCT/EP2023/080049

§ 371 Date: Apr. 17, 2025

(30) Foreign Application Priority Data
Oct. 28, 2022 (EP) 22204298.8

(51) Int. Cl. A61K 8/98; A61Q 17/04

(52) U.S. Cl. CPC A61K 8/987; A61Q 17/04; A61K 2800/592

(57) ABSTRACT

The present invention relates to emulsions containing at least one UVA sunscreen active agent and having improved sun protection factor (SPF) properties, as well as methods of improving sun protection factor (SPF) properties.

The present invention relates to compositions containing at least one UVA sunscreen active agent and having improved sun protection factor (SPF) properties, as well as methods of improving sun protection factor (SPF) properties.

The use of compositions containing UV sunscreen active agents (also called sunscreens) is very common nowadays. The sunscreen active agents in such compositions protect keratinous material such as in particular the skin from the harm caused by UV radiation, including harm from both UVA and UVB rays.

UVA rays have the least energy among UV rays. These rays can cause skin cells to age and can cause some indirect damage to cells' DNA. UVA rays are mainly linked to long-term skin damage such as wrinkles, but they are also thought to play a role in some skin cancers. UVB rays have a shorter wavelength. UVB radiation is associated with skin burning but also with skin cancer. Unprotected exposure to UVA and UVB damages the DNA in skin cells, producing genetic defects, or mutations, that can lead to skin cancer and premature aging.

Thus, to get ultimate skin protection, there's a constantly increasing need for sunscreens exhibiting high UVA as well as UVB protection.

A standard measure for determining the amount of protection a composition containing sunscreen active agent provides against UV radiation is the sun protection factor (SPF). However, given the finite amount of approved sunscreen active agents worldwide, increasing SPF of compositions containing sunscreen active agents can be difficult.

Thus, there is an ongoing need for preferably natural ingredients which are able to increase the SPF of sunscreen active ingredients in compositions.

Surprisingly it has now been found that certain human milk oligosaccharides are able to significantly increase the SPF of a composition comprising certain UVA filter in combination with UVB- and/or broadband UV-filters.

Thus, in a first embodiment, the present invention relates to a composition for keratinous materials (for example, hair or skin) comprising at least one UVA filter selected from the group consisting of butyl methoxydibenzoyl methane and diethylamino hydroxybenzoyl hexyl benzoate and at least one sialylated human milk oligosaccharide. Preferably, the composition is in the form of an oil in water (O/W emulsion).

The present invention also relates to methods of increasing the sun protection factor (SPF) properties of a composition comprising at least one UVA filter selected from the group consisting of butyl methoxydibenzoyl methane and diethylamino hydroxybenzoyl hexyl benzoate, said method comprising adding at least one sialylated human milk oligosaccharide to the composition in an amount effective to increase the SPF properties of the composition.

The invention also relates to a use of a sialylated human milk oligosaccharide for increasing the sun protection factor (SPF) of a composition comprising at least one UVA filter selected from the group consisting of butyl methoxydibenzoyl methane and diethylamino hydroxybenzoyl hexyl benzoate.

The present invention also relates to methods of preparing compositions having improved sun protection factor (SPF) properties comprising adding at least one UVA filter selected from the group consisting of butyl methoxydibenzoyl methane and diethylamino hydroxybenzoyl hexyl benzoate and at least one sialylated human milk oligosaccharide to the composition during preparation of the composition. It is well understood that said improved properties are to be compared to a composition not comprising the sialylated human milk oligosaccharide with all the definitions and preferences as given herein.

The present invention further relates to methods of making a composition, said method comprising combining at least one UVA filter selected from the group consisting of butyl methoxydibenzoyl methane and diethylamino hydroxybenzoyl hexyl benzoate and at least one sialylated human milk oligosaccharide in said composition.

In accordance with the invention is also the use of the composition of the invention for protection of skin from the effects skin aging (especially for protection from UV-induced skin aging) such as in particular wrinkles and as a sun protection composition.

Finally, a subject-matter of the invention is a method for the cosmetic treatment of keratinous substances such as in particular the skin, wherein a composition as defined herein is applied to the said keratinous substances such as in particular to the skin. The method is in particular suitable to protect the skin against the adverse effects of UV-radiation such as in particular sun-burn and/or photoaging.

The terms ‘UV-filter’ respectively ‘sunscreen active agent’ are used interchangeable herein and refer to compounds absorbing light in the UVB and/or the UVA-range (also referred to UVA-, UVB- and broadband (i.e. UVA & UVB) filters). Preferably, such compounds have an E1/1 value (i.e. UV absorbance at a concentration of 1% at 1 cm thickness of absorbing layer at λₘₐₓ) of at least 150, more preferably of at least 180, most preferably of at least 190.

The term ‘keratinous materials’ means the skin (body, face, contour of the eyes, scalp), head hair, eyelashes, eyebrows, bodily hairs, nails and/or lips. Preferably, in all embodiments the keratinous material is the skin.

The term ‘human milk oligosaccharides’ (HMOs) refers to a family of structurally diverse unconjugated glycans that are highly abundant in and unique to human milk. Originally, HMOs were proposed to be prebiotic “bifidus factors,” or human milk glycans found to promote growth in Bifidobacterial species of the gut and found uniquely in the stool of breast-fed infants compared to formula fed infants.

Butyl methoxydibenzoylmethane (CAS No: 70356-09-1; abbreviated BMDBM) is also known as avobenzone and e.g. sold under the trade name “Parsol® 1789 by DSM Nutritional Products Ltd.

Diethylamino hydroxybenzoyl hexyl benzoate (CAS No: 302776-68-7; abbreviated DHHB), is also known as hexyl 2-[4-(diethylamino)-2-hydroxybenzoyl]benzoate and e.g. sold under the tradename Uvinul® A plus by BASF.

The SPF according to the present invention is understood to be measured by the in vitro SPF method as outlined in ISO 24443 (Determination of sunscreen UVA photoprotection in vitro, Jan. 6, 2012) and as exemplified in the examples).

HMOs are generally composed of the five monosaccharides glucose (Glc), galactose (Gal), N-acetylglucosamine (GlcNAc), fucose (Fuc) and sialic acid (Sia), with N-acetylneuraminic acid (Neu5Ac) as the predominant if not only form of Sia. More than two hundred different HMOs have been identified so far, amongst the most important ones being 3′Sialyllactose Sodium Salt (3′SL) and 6′Sialyllactose Sodium Salt (6′SL).

HMOs can be isolated from breast milk, or they can be produced chemically or biochemically. HMOs are available commercially from a variety of producers.

For the purpose of the present invention the source of the sialylated HMO is not essential. It is clear that HMOs from different sources can be used.

Particularly preferred sialylated HMO's according to the present invention are sialyllactoses as well as salts thereof (preferably the sodium salts) such as in particular 3′sialyllactose (3′SL), 6′sialyllactose (6′SL) as well as the respective sodium salts thereof (CAS No's: 35890-39-2 (3′sialyllactose); 128596-80-5 (3′sialyllactose sodium salt); 35890-39-2 (6′sialyllactose); 157574-76-0 (6′sialyllactose sodium salt)).

The total amount of the HMO(s) according to the present invention in the compositions according to the present invention is preferably at least 0.01 wt.-%, more preferably at least 0.1 wt.-%, even more preferably at least 0.2 wt.-%, such as at least 0.3 wt.-% or at least 0.5 wt.-%, based on the total weight of the composition. In particular the total amount of the at least one HMO(s) according to the present invention in the compositions according to the present invention is preferably selected in the range from 0.01 to 10 wt.-%, more preferably in the range from 0.1 to 7.5 wt.-%, most preferably in the range from 0.2 to 5 wt.-%, based on the total weight of the composition. Further suitable ranges are from 0.25 to 5 wt.-%, from 0.5 to 4 wt.-%, from 0.1 to 3 wt.-%, from 0.25 to 3 wt.-% and from 0.3 to 3 wt.-%. Particularly preferred ranges according to the present invention are from 0.1 to 5 wt.-%, more preferably from 0.25 to 3 wt.-%, such as from 0.25 to 1.5 wt.-% or 0.5 to 5 wt.-%, or 0.5 to 4 wt.-%.

It is well understood, that the compositions according to the present invention may comprise one or more sialylated HMO's. Preferably, however, in all embodiments of the present invention the compositions according to the present invention comprise one or two, more preferably solely one sialylated HMO, such as most preferably solely one HMO selected from 3′SL and 6′SL, in particular in the form of their sodium salts. The most preferred sialylated HMO in all embodiments of the present invention is 6′SL (in the form of its sodium salt) as it leads to a particular pronounced increase in the SPF.

In all embodiments of the present invention, preferably the total amount of UVA filters selected from the group of butyl methoxydibenzoyl methane and diethylamino hydroxybenzoyl hexyl benzoate is selected in the range from 0.5 to 10 wt.-%, preferably in the range from 1 to 7.5 wt.-%, most preferably in the range from 2 to 6 wt.-%, based on the total weight of the composition.

Advantageously, in all embodiments of the present invention, the amount of butyl methoxydibenzoylmethane in the compositions according to the present invention is advantageously selected in the range from 0.4 to 10 wt.-%., preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 2 to 5 wt.-%, from 3 to 5 wt.-% or from 3.5 to 5 wt.-%, based on the total weight of the composition.

Further advantageously, in all embodiments of the present invention, the amount of diethylamino hydroxybenzoyl hexyl benzoate in the compositions according to the present invention is advantageously selected in the range from 0.5 to 10 wt.-%., preferably in the range from 0.5 to 9 wt.-%, 0.5 to 8 wt.-%, 0.5 to 7 wt.-%, 0.5 to 6 wt.-%, 0.5 to 5 wt.-%, 0.5 to 4 wt.-%, 0.5 to 3 wt.-%, 1 to 10 wt.-%, 1 to 9 wt.-%, 1 to 8 wt.-%, 1 to 7 wt.-%, 1 to 6 wt.-%, 1 to 5 wt.-%, 2 to 5 wt.-%, 3 to 5 wt.-%, such as for instance in the range from 2.5 to 6 wt.-%, from 3 to 6 wt.-%, from 4 to 6 wt.-% or from 3.5 to 5.5 wt.-%, based on the total weight of the composition.

Preferably, in all embodiments of the present invention, the compositions according to the present invention further comprises at least one, preferably at least two, most preferably at least three organic UVB and/or broadband sunscreen active agents.

Even more preferably, in all embodiments of the present invention combinations of one or more, preferably of two or more, most preferably of three and more different organic UVB and/or broadband filters are used in combination with butyl methoxydibenzoyl methane and/or diethylamino hydroxybenzoyl hexyl benzoate, such as combinations from 1 to 10, from 1 to 7 or from 1 to 5, from 1 to 4, from 1 to 3, from 2 to 10, from 2 to 7 or from 2 to 5 different UVB and/or broadband filters.

Advantageously the one or more, preferably the two or more, more preferably the three or more, even more preferably the four and more different UVB and/or broadband filters are selected from the group of organic UVB and/or broadband UV-filters with all the preferences and definitions as given herein.

Preferred organic UVB filters absorb radiation in the 280 to 320 nm region of the ultraviolet spectrum. Examples of preferred UVB absorbers include cinnamates, diphenylacrylates, benzalmalonates, triazines and salicylates.

Preferred organic broadband UV filters provide substantial broad-spectrum protection from both UVA and UVB rays. Examples of preferred broadband absorbers include mircronized organic UV-filters such as methylene bis-benzotriazolyl tetramethylbutylphenol.

Said organic UVB and/or broadband filter(s) can be soluble in water, soluble in non-aqueous material such as oils, and/or insoluble.

Preferably, in all embodiments of the present invention the total amount of all sunscreen active agent(s) (including butyl methoxydibenzoyl methane and/or diethylamino hydroxybenzoyl hexyl benzoate) in the compositions according to the invention is selected in the range from about 1 to about 50 wt.-% with respect to the total weight of the composition, preferably from about 3 to about 40 wt.-% with respect to the total weight of the composition, more preferably from about 5 to about 30 wt.-%, from about 10 to 25 wt.-% or from about 15 to 25 wt.-%, with respect to the total weight of the composition, including all ranges and subranges therebetween.

In another advantageous embodiment, the total amount of all sunscreen active agent(s) is selected in the range from about 5 to about 30 wt.-%, from about 7.5 to 25 wt.-% or from about 10 to 25 wt.-%, with respect to the total weight of the composition, including all ranges and subranges therebetween.

It is also preferred in all embodiments of the present invention, that the amount of all UV filters present in the composition according to the present invention is selected such that the SPF of the final composition (in vitro SPF measured as outlined in ISO 24443 Determination of sunscreen UVA photoprotection in vitro, Jan. 6, 2012, as exemplified in the examples) is at least 10, preferably at least 15, more preferably at least 20.

Organic UV-filters particular useful to be used in combination with butyl methoxydibenzoyl methane and/or diethylamino hydroxybenzoyl hexyl benzoate herein include anthranilates; cinnamic derivatives; salicylic derivatives; camphor derivatives; triazine derivatives, such as those disclosed in Patent Applications U.S. Pat. No. 4,367,390, EP 863 145, EP 517 104, EP 570 838, EP 796 851, EP 775 698, EP 878 469, EP 933 376, EP 507 691, EP 507 692, EP 790 243 and EP 944 624; benzophenone derivatives; β,β-diphenylacrylate derivatives; benzotriazole derivatives; benzalmalonate derivatives; benzimidazole derivatives; imidazolines; bisbenzoazolyl derivatives as disclosed in Patents EP 669 323 and U.S. Pat. No. 2,463,264; p-aminobenzoic acid (PABA) derivatives; methylenebis(hydroxyphenylbenzotriazole) derivatives as disclosed in Applications U.S. Pat. Nos. 5,237,071 and 5,166,355, GB 2 303 549, DE 197 26 184 and EP 893 1 19; screening polymers and screening silicones, such as those disclosed in particular in Application WO 93/04665; dimers derived from α-alkylstyrene, such as those disclosed in Patent Application DE 198 55 649; 4,4-diarylbutadienes as disclosed in Applications EP 0 967 200, DE 197 46 654, DE 197 55 649, EP-A-1 008 586, EP 1 133 980 and EP 133 981; and their mixtures.

By way of illustration, mention may be made, as additional sunscreen active agents to be used next to butyl methoxydibenzoyl methane and/or diethylamino hydroxybenzoyl hexyl benzoate, UV-filters which are generally active in the UVB or broadband regions, denoted below under their INCI names, of: p-aminobenzoic acid (PABA) derivatives, in particular PABA, ethyl PABA, ethyl dihydroxypropyl PABA, ethylhexyl dimethyl PABA, glyceryl PABA or PEG-25 PABA, salicylic derivatives, in particular homosalate (HMS), ethylhexyl salicylate (EHS), dipropylene glycol salicylate, or TEA salicylate; isopropyl dibenzoylmethane; cinnamic derivatives, in particular ethylhexyl methoxycinnamate (OMS), isopropyl methoxycinnamate, isoamyl methoxycinnamate, cinoxate, DEA methoxycinnamate, diisopropyl methyl cinnamate, or glyceryl ethylhexanoate dimethoxycinnamate, β,β-diphenylacrylate derivatives, in particular octocrylene (OC) or etocrylene, benzophenone, in particular benzophenone-1, benzophenone-2, benzophenone-3 or oxybenzone, benzophenone-4, benzophenone-5, benzophenone-6, benzophenone-8, benzophenone-9, benzophenone-12, benzylidene camphor derivatives, in particular 3-benzylidene camphor, 4-methylbenzylidene camphor, benzylidene camphor sulphonic acid, camphor benzalkonium methosulphate, terephthalylidene dicamphor sulphonic acid, or polyacrylamidomethyl benzylidene, benzimidazole derivatives, in particular phenylbenzimidazole sulphonic acid (HS), or disodium phenyl dibenzimidazole tetrasulphonate, triazine derivatives, in particular bis-ethylhexyloxyphenol methoxyphenyl triazine (BEMT), ethylhexyl triazone (EHT), diethylhexyl butamido triazone (DBT), or 2,4,6-tris(diisobutyl 4′-amino-benzalmalonate)-s-triazine, benzotriazole derivatives, in particular drometrizole trisiloxane or insoluble organic sunscreen active agents such as methylene bisbenzotriazolyl tetramethylbutylphenol (MBBT) and tris-biphenyl triazine (A2B), which are generally sold in the form of aqueous dispersions of the active UV filter e.g. under the tradename PARSOL® MAX (MBBT) from DSM Nutritional Products or Tinosorb® A2B (A2B) from BASF, anthranilic derivatives in particular menthyl anthranilate, imidazoline derivatives, in particular ethylhexyl dimethoxybenzylidene dioxoimidazoline propionate, benzalmalonate derivatives, in particular polyorganosiloxane comprising benzalmalonate functional groups (e.g. commercially available under the INCI name: polysilicone-15), 4,4-diarylbutadiene derivatives, in particular 1,1′-dicarboxy (2,2′-dimethylpropyl)-4,4-diphenylbutadiene, and their mixtures.

Particular preferred organic UVB and/or broadband filter(s) to be used in combination with butyl methoxydibenzoyl methane and/or diethylamino hydroxybenzoyl hexyl benzoate according to the present invention include homosalate (e.g. sold under the trade name “PARSOL® HMS”), ethylhexyl salicylate (e.g. sold under the trade name “PARSOL® EHS”), polysilicone-15 (sold in particular under the trade name “PARSOL® SLX”), ethylhexyl methoxycinnamate (e.g. sold under the trade name “PARSOL® MCX”), octocrylene (e.g. sold under the trade name “PARSOL® 340”), phenylbenzimidazole sulphonic acid (e.g. sold under the trade name “PARSOL® HS”) or a salt thereof, e.g. being formed during the preparation of the composition with a neutralization agent such as triethanolamine of sodium hydroxide, bis-ethylhexyloxyphenol methoxyphenyl triazine (e.g. sold under the trade name “PARSOL® SHIELD”), ethylhexyl triazone (e.g. sold under the trade name “PARSOL® EHT”), diethylhexyl butamido triazone (e.g. commercially available under the trade name Uvasaorb HEB), methylene bisbenzotriazolyl tetramethylbutylphenol (e.g. sold under the trade name “PARSOL® MAX”), tris-biphenyl triazine (e.g. sold under the trade name Uvinul A2B), and their mixtures.

Most preferred additional organic UVB and/or broadband filters to be used in combination with butyl methoxydibenzoyl methane and/or diethylamino hydroxybenzoyl hexyl benzoate according to the present invention are selected from the group consisting of homosalate, ethylhexyl salicylate, polysilicone-15, ethylhexyl methoxycinnamate, octocrylene, phenylbenzimidazole sulphonic acid or a salt thereof, bis-ethylhexyloxyphenol methoxyphenyl triazine, ethylhexyl triazone, diethylhexyl butamido triazone, methylene bisbenzotriazolyl tetramethylbutylphenol, tris-biphenyl triazine, butyl methoxydibenzoylmethane, diethylamino hydroxybenzoyl hexyl benzoate, preferably of ethylhexyltriazone, octocrylene, homosalate, ethylhexyl methoxycinnamate and polysilicone-15 as well as any mixtures thereof.

In all embodiments of the present invention, the (total) amount of the organic UVB and/or broadband filters in the compositions according to the present invention is selected in the range from 0.5 to 30 wt.-%, more preferably in the range from 1 to 25 wt.-%, and most preferably in the range from 1 to 25 wt.-%, such as from 1 to 15 wt.-%, based on the total weight of the composition. Further suitable amounts are selected in the range from 5 to 30 wt.-%, 7.5 to 30 wt.-%, 10 to 30 wt.-%, from 5 to 25 wt.-%, 7.5 to 25 wt.-% or from 10 to 25 wt.-%, based on the total weight of the composition.

In all embodiments of the present invention, the amount of ethylhexyl salicylate in the compositions according to the present invention is advantageously selected in the range from 0.5 to 10 wt.-%., preferably in the range from 0.5 to 9 wt.-%, 0.5 to 8 wt.-%, 0.5 to 7 wt.-%, 0.5 to 6 wt.-%, 0.5 to 5 wt.-%, such as for instance in the range from 1 to 10 wt.-%, from 2 to 10 wt.-%, from 3 to 10 wt.-% or from 2 to 7.5 wt.-%, based on the total weight of the composition.

In all embodiments of the present invention, the amount of polysilicone-15 in the compositions according to the present invention is advantageously selected in the range from 0.4 to 10 wt.-%., preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 1 to 3 wt.-%, from 1 to 2.5 wt.-% or from 1.5 to 2.5 wt.-%, based on the total weight of the composition.

In all embodiments of the present invention, the amount of ethylhexyl methoxycinnamate in the compositions according to the present invention is advantageously selected in the range from 0.5 to 10 wt.-%., preferably in the range from 0.5 to 9 wt.-%, 0.5 to 8 wt.-%, 0.5 to 7 wt.-%, 0.5 to 6 wt.-%, 0.5 to 5 wt.-%, such as for instance in the range from 1 to 10 wt.-%, from 2 to 10 wt.-%, from 3 to 10 wt.-% or from 2 to 7.5 wt.-%, based on the total weight of the composition.

In all embodiments of the present invention, the amount of octocrylene in the compositions according to the present invention is advantageously selected in the range from 0.5 to 15 wt.-%., preferably in the range from 0.5 to 10 wt.-%, 0.5 to 8 wt.-%, 1 to 10 wt.-%, 2 to 10 wt.-%, 3 to 10 wt.-%, such as for instance in the range from 5 to 10 wt.-%, from 6 to 10 wt.-% or from 7 to 10 wt.-%, based on the total weight of the composition.

In all embodiments of the present invention, the amount of phenylbenzimidazole sulphonic acid or a salt thereof in the compositions according to the present invention is advantageously selected in the range from 0.5 to 5 wt.-%., preferably in the range from 0.5 to 4 wt.-%, 0.5 to 3 wt.-%, 1 to 2.5 wt.-%, 1 to 3 wt.-% or 1 to 4 wt.-%, based on the total weight of the composition.

In all embodiments of the present invention, the amount of bis-ethylhexyloxyphenol methoxyphenyl triazine in the compositions according to the present invention is advantageously selected in the range from 0.4 to 10 wt.-%., preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 1 to 3 wt.-%, from 1 to 2.5 wt.-% or from 1.5 to 2.5 wt.-%, based on the total weight of the composition.

In all embodiments of the present invention, the amount of ethylhexyl triazone in the compositions according to the present invention is advantageously selected in the range from 0.4 to 10 wt.-%., preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 1 to 3 wt.-%, from 1 to 2.5 wt.-% or from 1.5 to 2.5 wt.-%, based on the total weight of the composition.

In all embodiments of the present invention, the amount of diethylhexyl butamido triazone in the compositions according to the present invention is advantageously selected in the range from 0.4 to 10 wt.-%., preferably in the range from 0.4 to 9 wt.-%, 0.4 to 8 wt.-%, 0.4 to 7 wt.-%, 0.4 to 6 wt.-%, 0.4 to 5 wt.-%, 0.4 to 4 wt.-%, 0.4 to 3 wt.-%, 0.5 to 3 wt.-%, 0.8 to 9 wt.-%, 0.8 to 8 wt.-%, 0.8 to 7 wt.-%, 0.8 to 6 wt.-%, 0.8 to 5 wt.-%, 0.8 to 4 wt.-%, 0.8 to 3 wt.-%, such as for instance in the range from 1 to 5 wt.-%, from 1 to 3 wt.-%, or from 1.5 to 3 wt.-%, based on the total weight of the composition.

In all embodiments of the present invention, the amount of methylene bisbenzotriazolyl tetramethylbutylphenol (based on a dispersion of approx. 45-55 wt.-% of MBBT as commercially available e.g. under the tradename PARSOL MAX) in the compositions according to the present invention is advantageously selected in the range from 0.5 to 10 wt.-%, preferably in the range from 1 to 10 wt.-%, more preferably in the range from 2 to 10 wt.-%, most preferably in the range from 2 to 6 wt.-% or from 3 to 6 wt.-%, based on the total weight of the composition.

Particularly preferred organic UV-filter combinations in all embodiments of the present invention encompass the combination of DHHB, OMC, polysilicone-15, and DBT as well as BMDBM, EHT, OC and HMS or _DHHB, EHT, BEMPT, OMC, poysilicone-15, DBT and MBBT as exemplified.

In one preferred embodiment, the compositions according to the present invention only comprise organic UV-filters.

Further UVB and/or broadband filter(s), which can be present in the compositions of the present invention are inorganic sunscreen active agents (inorganic UV-filters) such as for example, physical blockers such as titanium dioxide or zinc oxide.

Suitable titanium dioxide UV-filters for use according to the present invention including any pre-dispersions thereof are e.g. available as PARSOL® TX (INCI: titanium dioxide, silica, dimethicone) at DSM Nutritional Products Ltd., Micro Titanium dioxide MT-01 at Tayca or TTO-551 at Ishihara Sangyo Kaisha. Also suitable is double coated titanium dioxide having an inner alumina coating and an outer simethicone coating e.g. commercially available as Eusolex T-2000 at EMD chemicals Inc./Rona.

Suitable zinc oxide UV-filters for use according to the present invention including any pre-dispersions thereof are e.g. available from BASF as Z-Cote or Z-Cote HP1 (2% dimethicone coating), from Tayca as MZ-505S (5% methicone coating), or from DSM Nutritional Products Ltd as PARSOL® ZX (2-3.5% triethoxycaprylylsilane coating).

If nothing else is stated in this specification any given parts and percentages are per weight and based on the total weight of the composition.

Preferably, the sialylated human milk oligosaccharide and the total amount of sunscreen active agent(s) are present in the compositions and methods of the invention in a weight ratio between 1:50 and 1:1, preferably between 1:40 and 1:7.5, and preferably between 1:30 and 1:5, such as 1:25 to 1:15. Preferably more sunscreen active agent(s) are present than HMO's on a weight basis.

As the compositions according to the invention are intended for topical application, it is well understood that they comprise a physiologically acceptable medium, i.e. a medium compatible with keratinous substances, such as the skin, mucous membranes, and keratinous fibres. In particular, the physiologically acceptable medium is a cosmetically acceptable carrier.

The term ‘cosmetically acceptable carrier’ as used herein refers to all carriers and/or excipients and/or diluents conventionally used in topical cosmetic compositions such as in particular in skin care preparations.

The exact amount of carrier will depend upon the actual level of the UV filters and any other optional ingredients that one of ordinary skill in the art would classify as distinct from the carrier (e.g., other active ingredients).

In an advantageous embodiment, the compositions according to the present invention comprise from 50% to 99%, preferably from 60% to 98%, more preferably from 70% to 98%, such as in particular from 80% to 95% of a carrier, based on the total weight of the composition.

In a particular advantageous embodiment, the carrier in all embodiments of the present invention consists furthermore of at least 30 wt. %, more preferably of at least 40 wt.-%, most preferably of at least 45 wt.-% of water, such as in particular of 50 to 90 wt.-%, from 50 to 80 wt.-%, from 50 to 79 wt.-% or from 50 to 60 wt.-% of water.

In particular, the compositions according to the present invention are cosmetic or pharmaceutical compositions, preferably cosmetic (non-therapeutic) compositions.

In one embodiment, the compositions according to the present invention are applied to mammalian keratinous tissue such as in particular to human skin or the human scalp and hair.

The term “cosmetic composition” as used in the present application refers to cosmetic compositions as defined under the heading “Kosmetika” in Rompp Lexikon Chemie, 10^{th} edition 1997, Georg Thieme Verlag Stuttgart, New York as well as to cosmetic compositions as disclosed in A. Domsch, “Cosmetic Compositions”, Verlag für chemische Industrie (ed. H. Ziolkowsky), 4^{th} edition, 1992.

Preferred compositions according to the invention are skin care preparations, decorative preparations, and functional preparations.

Examples of skin care preparations are, in particular, light protective preparations, anti-ageing preparations, preparations for the treatment of photo-ageing, body oils, body lotions, body gels, treatment creams, skin protection ointments, skin powders, moisturizing gels, moisturizing sprays, face and/or body moisturizers, skin-tanning preparations (i.e. compositions for the artificial/sunless tanning and/or browning of human skin), for example self-tanning creams as well as skin lightening preparations.

Examples of decorative preparations are, in particular, lipsticks, eye shadows, mascaras, dry and moist make-up formulations, rouges and/or powders.

Examples of functional preparations are cosmetic or pharmaceutical compositions containing active ingredients such as hormone preparations, vitamin preparations, vegetable extract preparations, anti-ageing preparations, and/or antimicrobial (antibacterial or antifungal) preparations without being limited thereto.

In a particular embodiment, the compositions according to the invention are light-protective preparations (sun care products, sunscreens), such as sun protection milks, sun protection lotions, sun protection creams, sun protection oils, sun blocks or day care creams with a SPF. Of particular interest are sun protection creams, sun protection lotions and sun protection milks.

The compositions of the invention (including the carrier) may comprise conventional adjuvants and additives, such as preservatives/antioxidants, fatty substances/oils, organic solvents, silicones, thickeners, softeners, emulsifiers, antifoaming agents, aesthetic components such as fragrances, surfactants, fillers, anionic, cationic, nonionic or amphoteric polymers or mixtures thereof, propellants, acidifying or basifying agents, dyes, colorings/colorants, abrasives, absorbents, chelating agents and/or sequestering agents, essential oils, skin sensates, astringents, pigments or any other ingredients usually formulated into such compositions.

In accordance with the present invention, the compositions according to the invention may comprise further ingredients such as ingredients for skin lightening; tanning prevention; treatment of hyperpigmentation; preventing or reducing acne, wrinkles, lines, atrophy and/or inflammation; chelators and/or sequestrants; anti-cellulites and slimming (e.g. phytanic acid), firming, moisturizing and energizing, self-tanning, soothing agents, as well as agents to improve elasticity and skin barrier and carriers and/or excipients or diluents conventionally used in compositions.

If nothing else is stated, the excipients, additives, diluents, etc. mentioned in the following are suitable for compositions according to the present invention. The necessary amounts of the cosmetic and dermatological adjuvants and additives can, based on the desired product, easily be determined by the skilled person.

The additional ingredients can either be added to the oily phase, the aqueous phase or separately as deemed appropriate. The mode of addition can easily be adapted by a person skilled in the art.

Examples of cosmetic excipients, diluents, adjuvants, additives as well as active ingredients commonly used in the skin care industry which are suitable for use in the cosmetic compositions of the present invention are for example described in the International Cosmetic Ingredient Dictionary & Handbook by Personal Care Product Council, without being limited thereto.

The cosmetically active ingredients useful herein can in some instances provide more than one benefit or operate via more than one mode of action.

Of course, one skilled in this art will take care to select the above mentioned optional additional ingredients, adjuvants, diluents and additives and/or their amounts such that the advantageous properties intrinsically associated with the combination in accordance with the invention are not, or not substantially, detrimentally affected by the envisaged addition or additions.

Preferred compositions in all embodiments of the present invention are emulsions containing an oily phase and an aqueous phase such as in particular an O/W, W/O, Si/W, W/Si, O/W/O, W/O/W multiple or a pickering emulsions. The amount of the oily phase (i.e. the phase containing all oils and fats) present in such emulsions is preferably at least 10 wt.-%, such as in the range from 10 to 60 wt.-%, preferably in the range from 15 to 50 wt.-%, most preferably in the range from 15 to 45 wt.-%, based on the total weight of the composition.

According to one even more preferred embodiment, the compositions according to the present invention as outlined herein are O/W emulsions comprising an oily phase dispersed in an aqueous phase in the presence of an O/W emulsifier. The preparation of such O/W emulsions is well known to a person skilled in the art and illustrated in the examples.

In one advantageous embodiment, the O/W emulsifier is a phosphate ester emulsifier. Among the preferred phosphate ester emulsifier are C8-10 alkyl ethyl phosphate, C9-15 alkyl phosphate, C20-22 alkyl phosphate, ceteareth-2 phosphate, ceteareth-5 phosphate, ceteth-8 phosphate, ceteth-10 phosphate, cetyl phosphate, C6-10 pareth-4 phosphate, C12-15 pareth-2 phosphate, C12-15 pareth-3 phosphate, DEA-ceteareth-2 phosphate, DEA-cetyl phosphate, DEA-oleth-3 phosphate, potassium cetyl phosphate, deceth-4 phosphate, deceth-6 phosphate and trilaureth-4 phosphate. Particular preferred phosphate ester emulsifiers in all embodiments of the present invention are C8-C25 alkyl phosphates, such as in particular C20-22 alkyl phosphates and cetyl phosphates, and in particular the respective potassium salts thereof. Particular preferred phosphate ester emulsifier according to the invention are potassium cetyl phosphate e.g. commercially available as Amphisol® K at DSM Nutritional Products Ltd Kaiseraugst or C20-22 alkyl phosphate e.g. commercially available as Sensanov WR.

Further suitable O/W emulsifiers according to the present invention encompass PEG 30 Dipolyhydroxystearate, PEG-4 Dilaurate, PEG-8 Dioleate, PEG-40 Sorbitan Peroleate, PEG-7 Glyceryl Cocoate, PEG-20 Almond Glycerides, PEG-25 Hydrogenated Castor Oil, Glyceryl Stearate (and) PEG-100 Stearate, PEG-7 Olivate, PEG-8 Oleate, PEG-8 Laurate, PEG-60 Almond Glycerides, PEG-20 Methyl Glucose Sesquistearate, PEG-40 Stearate, PEG-100 Stearate, PEG-80 Sorbitan Laurate, Steareth-2, Steareth-12, Oleth-2, Ceteth-2, Laureth-4, Oleth-10, Oleth-10/Polyoxyl 10 Oleyl Ether, Ceteth-10, Isosteareth-20, Ceteareth-20, Oleth-20, Steareth-20, Steareth-21, Ceteth-20, Isoceteth-20, Laureth-23, Steareth-100, glycerylstearatcitrate, glycerylstearate (self-emulsifying), stearic acid, salts of stearic acid, polyglyceryl-3-methylglycosedistearate. Further suitable emulsifiers are sorbitan oleate, sorbitan sesquioleate, sorbitan isostearate, sorbitan trioleate, Lauryl Glucoside, Decyl Glucoside, Sodium Stearoyl Glutamate, Sucrose Polystearate and Hydrated Polyisobuten. Furthermore, one or more synthetic polymers may be used as an emulsifier. For example, PVP eicosene copolymer, acrylates/C10-30 alkyl acrylate crosspolymer, acrylates/steareth-20 methacrylate copolymer, PEG-22/dodecyl glycol copolymer, PEG-45/dodecyl glycol copolymer, and mixtures thereof.

Another particular suitable class of O/W emulsifiers are non-ionic self-emulsifying system derived from olive oil e.g. known as (INCI Name) cetearyl olivate and sorbitan olivate (Chemical Composition: sorbitan ester and cetearyl ester of olive oil fatty acids) sold under the tradename OLIVEM 1000.

Further suitable are commercially available polymeric emulsifiers such as hydrophobically modified polyacrylic acid such as Acrylates/C10-30 Alkyl Acrylate Crosspolymers which are commercially available under the tradename Pemulen® TR-1 and TR-2 by Lubrizol.

Another class of particularly suitable emulsifiers are polyglycerol esters or diesters of fatty acids also called polyglyceryl ester/diester (i.e. a polymer in which fatty acid(s) is/are bound by esterification with polyglycerine), such as e.g. commercially available at Evonik as Isolan GPS Name Polyglyceryl-4 [INCI Diisostearate/Polyhydroxystearate/Sebacate (i.e. diester of a mixture of isostearic, polyhydroxystearic and sebacic acids with Polyglycerin-4)] or Dehymuls PGPH available at BASF (INCI Polyglyceryl-2 Dipolyhydroxystearate).

Also suitable are polyalkylenglycolether such as Brij 72 (Polyoxyethylen(2)stearylether) or Brij 721 (Polyoxyethylene (21) Stearyl Ether e.g. available at Croda.

Particularly advantageous O/W emulsifiers according to the present invention are one or more of Polyglyceryl-3 Methylglucose Distearate, Lauryl Glucoside (and) Polyglyceryl-2 Dipolyhydroxystearate, Glyceryl Sterate Citrate, Sodium Cetearyl Sulfate, Cetearyl Glucoside; Polyglyceryl-6 Stearate (and) Polyglyceryl-6 Behenate, Cetearyl Olivate (and) Sorbitan Olivate, Arachidyl Alcohol (and) Behenyl Alcohol (and) Arachidyl Glucosides, Cetearyl Alcohol (and) Coco-Glucoside, Coco-Glucoside (and) Coconut Alcohol, PEG-100 Stearate (and) Glyceryl Stearate, Sodium Stearoyl Glutamate, Steareth-20, Steareth-21, Steareth-25, Steareth-2, Ceteareth-25 and Ceteareth-6 (all listed by their INCI names).

In one embodiment it is particularly preferred in accordance with the invention if the composition comprises a C8-C25 alkyl phosphate, preferably a C20-22 alkyl phosphate and/or a cetyl phosphate as emulsifier or at least as a co-emulsifier.

In another embodiment it is particularly preferred in accordance with the invention if the composition comprises Polyglyceryl-6 Stearate and/or Polyglyceryl-6 Behenate as emulsifier, preferably in the presence of a cetyl phosphate, more preferably potassium cetyl phosphate as a co-emulsifier.

In a further embodiment it is particularly preferred in accordance with the invention if the composition comprises Polyglyceryl-3 Methylglucose Distearate as emulsifier.

The at least one O/W respectively Si/W emulsifier is preferably used in an amount of 0.5 to 10 wt. % such as in particular in the range of 0.5 to 5 wt.-% such as most in particular in the range of 0.5 to 4 wt.-% or in the range of 0.5 to 3 wt.-%, based on the total weight of the composition.

If the phosphate ester emulsifier with all the definitions and preferences as given herein is used as a co-emulsifier, the amount is preferably selected in the range of 0.1 to 1 wt. % such as in particular in the range of 0.25 to 0.75 wt.-% such as most in particular in the range of 0.3 to 0.7 wt.-% or in the range of 0.4 to 0.6 wt.-%, based on the total weight of the composition.

Suitable W/O- or W/Si-emulsifiers according to the present invention are polyglyceryl-2-dipolyhydroxystearat, PEG-30 dipolyhydroxystearat, cetyl dimethicone copolyol, polyglyceryl-3 diisostearate polyglycerol esters of oleic/isostearic acid, polyglyceryl-6 hexaricinolate, polyglyceryl-4-oleate, oleate/PEG-8 propylene glycol cocoate, magnesium stearate, sodium stearate, potassium laurate, potassium ricinoleate, sodium cocoate, sodium tallowate, potassium castorate, sodium oleate, and mixtures thereof. Further suitable W/Si-emulsifiers Lauryl Polyglyceryl-3 are Polydimethylsiloxyethyl Dimethicone and/or PEG-9 Polydimethylsiloxyethyl Dimethicone and/or Cetyl PEG/PPG-10/1 Dimethicone and/or PEG-12 Dimethicone Crosspolymer and/or PEG/PPG-18/18 Dimethicone. The at least one W/O emulsifier is preferably used in an amount of about 0.001 to 10 wt.-%, more preferably in an amount of 0.2 to 7 wt.-% with respect to the total weigh of the composition.

The compositions according to the present invention furthermore advantageously contain at least one co-surfactant such as e.g. selected from the group of mono- and diglycerides and/or fatty alcohols. The co-surfactant is generally used in an amount selected in the range of 0.1 to 10 wt.-%, such as in particular in the range of 0.5 to 7 wt.-%, such as most in particular in the range of 1 to 5 wt.-%, based on the total weight of the composition. Particular suitable co-surfactants are selected from the list of alkyl alcohols such as cetyl alcohol (Lorol C16, Lanette 16), cetearyl alcohol (Lanette O), stearyl alcohol (Lanette 18), behenyl alcohol (Lanette 22), polyhydroxystearic acid, glyceryl stearate, glyceryl myristate (Estol 3650), hydrogenated coco-glycerides (Lipocire Na10) as well as mixtures thereof.

In all embodiments of the present invention, it is particular advantageous if the composition also comprises cetearyl alcohol and/or glycerylstearate.

Advantageous embodiments of the composition of the present invention also include those wherein the composition comprises one or more oils selected from butylene glycol dicaprylate/dicaprate, phenethyl benzoate, C₁₂-C₁₅ alkyl benzoate, dibutyl adipate, diisopropyl sebacate, dicaprylyl carbonate, di-C₁₂₋₁₃ alkyl tartrates, diethylhexyl syringylidene malonates, hydrogenated castor oil dimerates, triheptanoin, C₁₂₋₁₃ alkyl lactates, C₁₆₋₁₇ alkyl benzoates, propylheptyl caprylates, caprylic/capric triglycerides, diethylhexyl 2,6-naphthalates, octyldodecanol, ethylhexyl cocoates, and phenethyl alcohol. Preferably, the composition according to the present invention comprises as oil(s) caprylic/capric triglycerides, dibutyl adipate, phenylethyl benzoate, butylene glycol, phenethyl benzoate, phenethyl alcohol, C₁₂-C₁₅ alkylbenzoate, diisopropyl sebacate, as well as mixtures thereof.

In a still further advantageous aspect of the invention, the compositions of the present invention further comprise a preservative and/or a preservative booster, preferably selected from the group consisting of ethanol, phenoxyethanol, sodium benzoate, potassium sorbate, phenethyl alcohol, hydroxyactophenone, ethylhexylglycerin, hexylglycerin, glyceryl caprylate, 1,3-propanediol, 1,2-pentanediol, 1,2-hexanediol, 1,2-octanediol (caprylyl glycol), 1,2-decanediol and 2-methyl-1,3-propanediol as well as mixtures thereof, most preferably selected from the group of phenoxyethanol and ethylhexyl glycerin as well as mixtures thereof. When present, the preservative respectively the preservative booster is preferably used in an amount of 0.01 to 5 wt. %, more preferably in an amount of 0.05 to 4 wt.-%, most preferably in an amount of 0.1 to 3.0 wt.-%, based on the total weight of the composition.

It is advantageous in accordance with the invention if the preparation comprises one or more alkanediols from the group 1,3-propanediol, 1,2-propanediol, 1,2-pentanediol, 1,2-hexanediol, 1,2-octanediol, 1,2-decanediol and 2-methyl-1,3-propanediol. When present, the one or more alkanediols is preferably used in an amount of 0.1 to 7 wt. %, more preferably in an amount of 0.5 to 7 wt.-%, most preferably in an amount of 1.0 to 5.0 wt.-%, based on the total weight of the composition.

It is further advantageous in accordance with the invention if the emulsion composition of the invention comprises phenoxyethanol, ethylhexylglycerin, glyceryl caprylate and/or phenethyl alcohol, preferably phenoxyethanol and/or ethylhexylglycerin and/or glyceryl caprylate and/or phenethyl alcohol, most phenoxyethanol and ethylhexylglycerin and/or glyceryl caprylate.

In another advantageous aspect, the compositions according to the present invention are free of any parabenes, benzethoniumchlorid, piroctone olamine, lauroylarginat, methylisothiazolinon, chlormethylisothiazolinon, bronopol, benzalkoniumchloride, formaldehyde releasing compounds, salicylic acid, triclosan, DMDM hydantoin, chlorphenesin and IPBC (lodopropinylbutyl carbamate).

The compositions according to the invention may further contain one or more emollients which soothe and soften the skin. As an example, the emollient may be silicone (dimethicone, cyclomethicone), vegetable oils (grape seed, sesame seed, jojoba, etc.), butters (cocoa butter, shea butter), and petrolatum derivatives (petroleum jelly, mineral oil).

In another aspect, the composition of the invention may comprise one or more fragrances selected from limonene, citral, linalool, alpha-isomethylionone, geraniol, citronellol, 2-isobutyl-4-hydroxy-4-methyltetrahydropyran, 2-tert-pentylcyclohexyl acetate, 3-methyl-5-phenyl-1-pentanol, 7-acetyl-1,1,3,4,4,6-hexamethyltetralin, adipic diester, cinnamal, amyl salicylate, alpha-amylcinnamaldehyde, alpha-methylionone, butylphenylmethylpropional, cinnamal, amylcinnamyl alcohol, anise alcohol, benzoin, benzyl alcohol, benzyl benzoate, benzyl cinnamate, benzyl salicylate, bergamot oil, bitter orange oil, butylphenylmethylpropional, cardamom oil, cedrol, cinnamal, cinnamyl alcohol, citronellyl methylcrotonate, citrus oil, coumarin, diethyl succinate, ethyllinalool, eugenol, Evernia furfuracea extract, Evernia prunastri extract, farnesol, guaiacwood oil, hexylcinnamal, hexyl salicylate, hydroxycitronellal, lavender oil, lemon oil, linalyl acetate, mandarin oil, menthyl PCA, methyl hexadecan, nutmeg oil, rosemary oil, sweet orange oil, terpineol, tonkabean oil, triethyl citrate, vanillin.

The composition of the invention may advantageously comprise moisturizers. Moisturizers are compounds or mixtures of compounds which give cosmetic compositions the quality, after application to or distribution on the skin surface, of reducing the loss of moisture of the stratum corneum (also called transepidermal water loss (TEWL)) and/or of positively influencing the hydration of the stratum corneum.

Non-limiting examples of advantageous moisturizers for use in the present invention include glycerol, lactic acid and/or lactates, especially sodium lactate, butylene glycol, propylene glycol, 1,3-propanediol, biosaccharide gum-1, Glycine soya, ethylhexyloxyglycerol, pyrrolidonecarboxylic acid, and urea. Of further advantage, in particular, is the use of polymeric moisturizers from the group of the polysaccharides which are water-soluble and/or swellable in water and/or gellable with the aid of water. Especially advantageous, for example, are hyaluronic acid, chitosan and/or a fucose-rich polysaccharide which is registered in Chemical Abstracts under the registry number 178463-23-5 and is available, for example, under the Fucogel®1000 name from the company SOLABIA S.A. Moisturizers may also be used advantageously as active antiwrinkle ingredients for protection from changes to the skin of the kind occurring in skin aging, for example.

Particularly advantageous moisturizers to be used in the compositions according to the present invention are glycerin, sodium hyaluronate, saccharide isomerate and/or 1,3-propanediol, more preferably in a total amount selected in the range of 0.1 to 5 wt.-%, more preferably in the range from to 0.1 to 3 wt.-%, even more preferably in the range of 0.5 to 3 wt.-%, based on the total weight of the composition.

The cosmetic compositions of the invention may further comprise advantageously, although not mandatorily, fillers which have the effect, for example, of further improving the sensorial and cosmetic properties of the formulations and evoking or intensifying a velvety or silky skin sensation, for example. Advantageous fillers in the sense of the present invention are starch and starch derivatives (such as tapioca starch, distarch phosphate, aluminum or sodium starch octenylsuccinate, and the like, for example), Valvance pigments which have neither primarily UV-filter effect nor coloring effect (such as Valvance Touch 210 or 250 for example) and/or Aerosils® (CAS No. 7631-86-9) and/or talc and/or polyethylene, nylon, and silica dimethyl silylate.

The water phase of the compositions of the invention may advantageously comprise customary cosmetic auxiliaries, such as, for example, alcohols, particularly those of low C number, preferably ethanol and/or isopropanol, or polyols of low C number, and also ethers thereof, preferably propylene glycol, glycerol, electrolytes, self-tanning agents, and also, in particular, one or more thickeners, which may be advantageously selected from the group of silicon dioxide, aluminum silicates, polysaccharides and/or derivatives thereof, e.g., hyaluronic acid, xanthan gum, hydroxypropylmethylcellulose, particularly advantageously from the group of polyacrylates, preferably a polyacrylate from the group referred to as Carbopols, examples being carbopols of types 980, 981, 1382, 2984, and 5984, in each case individually or in combination. Further thickeners advantageous in accordance with the invention are those having the INCI designation Acrylates/C10-30 Alkyl Acrylate Crosspolymer (e.g., Pemulen TR 1, Pemulen TR 2, Carbopol 1328 from Lubrizol) and also Aristoflex AVC (INCI: Ammonium Acryloyldimethyltaurate/VP Copolymer) as well as Simugel NS (INCI: Hydroxyethyl Acrylate/Sodium Acryloyldimethyl Taurate Copolymer & Squalane & Polysorbate 60).

It is further preferred in accordance with the invention if the composition according to the present invention comprises a thickener. Suitable thickeners to be used according to the present invention encompass xanthan gum, crosslinked acrylate/C10-C30 alkyl acrylate polymer, hydroxyethyl acrylate/sodium acryloyldimethyl taurate copolymer, and/or vinylpyrrolidone/hexadecane copolymer, preferably xanthan gum and/or hydroxyethyl acrylate/sodium acryloyldimethyl Taurate, most preferably xanthan gum.

The amount of xanthan gum, if present is preferably selected in the range of 0.1 to 1 wt.-%, more preferably in the range of 0.15 to 0.75 wt.-%, most preferably in the range of 0.2 to 0.4 wt.-%, based on the total weight of the composition.

The oil phase of the compositions of the invention may advantageously also comprise customary cosmetic auxiliaries, such as, for example, thickeners for the oil phase such as Dextrin Palmitate (Rheopearl KL2, Chiba Flour Milling), Dextrin Palmitate and C15-19 Alkane (TILAMAR Fits, DSM), Polyurethane-79 and Caprylic/Capric Triglyceride (Oilkemia 5S, Lubrizol), Ethylene/Propylene/Styrene Copolymer and Butylene/Ethylene/Styrene Copolymer and Pentaerythrityl Tetra-di-t-butyl Hydroxyhydrocinnamate and Squalane (Veggi Gel, Aiglon), Dibutyl Lauroyl Glutamide and Dibutyl Ethylhexanoyl Glutamide and Octyldodecanol (AJK-OD246, Ajinomoto).

The amount of the thickener (e.g. the active thickening polymer), if present is preferably selected in the range of 0.1 to 15 wt.-%, more preferably in the range of 0.5 to 10 wt.-%, most preferably in the range of 1.0 to 8.0 wt.-%, based on the total weight of the composition.

The compositions of the invention manage with a surprisingly small total amount of UV-filters.

In another aspect, the composition may have an SPF of at least 15, preferably at least 20, most preferably of at least 30.

Advantageously in accordance with the invention, the composition of the invention comprises film formers. Film formers in the sense of the present invention are substances of various constitutions, and are characterized by the following properties: When a film former is dissolved in water or other suitable solvents, and when the solution is then applied to the skin, the film former, following evaporation of the solvent, forms a film which serves essentially to fix the UV-filters on the skin and thus to increase the water resistance of the product.

It is especially advantageous to select the film formers from the group of the polymers based on polyvinylpyrrolidone (PVP)

Particular preference is given to copolymers of vinylpyrrolidone, as for example the PVP hexadecane copolymer and the PVP eicosene copolymer, which are available under the trade names Antaron V216 and Antaron V220 from Ashland.

Likewise advantageous are further polymeric film formers, such as, for example, sodium polystyrene sulfonate, which is available under the trade name Flexan 130 from National Starch and Chemical Corp., and/or polyisobutene, available from Rewo under the trade name Rewopal PIB1000. Examples of further suitable polymers are polyacrylamides (Sepigel 305), polyvinyl alcohols, PVP, PVP/VA copolymers, polyglycols, acrylate/octylacrylamide copolymer (Dermacryl 79). Likewise advantageous is the use of hydrogenated castor oil dimer dilinoleate (CAS 646054-62-8, INCI Hydrogenated Castor Oil Dimer Dilinoleate), which can be acquired from Kokyu Alcohol Kogyo under the name Risocast DA-H, or else PPG-3 benzyl ether myristate (CAS 403517-45-3), which can be acquired under trade name Crodamol STS from Croda Chemicals.

The compositions according to the invention in general have a pH in the range of 3 to 10, preferably a pH in the range of 4 to 8 and most preferably a pH in the range of 4 to 7.5. The pH can easily be adjusted as desired with suitable acids such as e.g. citric acid or bases such as NaOH or suitable amines such as tromethamine according to standard methods in the art.

The amount of the cosmetic composition to be applied to the skin is not critical and can easily be adjusted by a person skilled in the art. Preferably the amount is selected in the range of 0.1 to 3 mg/cm² skin, such as preferably in the range of 0.1 to 2 mg/cm² skin and most preferably in the range of 0.5 to 2 mg/cm² skin.

The following examples are provided to further illustrate the compositions and effects of the present invention. These examples are illustrative only and are not intended to limit the scope of the invention in any way.

### EXAMPLES

### Short Description

The HMO's 3SL and 6SL were evaluated for their SPF boosting effect. For this purpose various sunscreens were prepared according to standard methods in the art as outlined in table 1a.

### In Vitro SPF Measurement

The in vitro SPF test was performed on 4 PMMA plates (HD6 plates from Helioscreen, 5 cm×5 cm, roughness of Sa=6 μm): 32.5 mg of the respective formulation (i.e. 1.3 mg/cm²) were applied homogenously onto the PMMA plates and dried for 30 minutes.

The in vitro SPF was determined using a Labsphere 2000 UV Transmittance Analyzer: each PMMA plate was measured 9 times at different points on the plate resulting in 36 data points. The result is calculated as the average of these 36 data points.

TABLE 1a
Formulation 1 & 2

| | | 1 | 2 |
|---|---|---|---|
| Tradename | INCI name | Conc. [wt.-%] | |
| Sensanov WR | C20-22 Alkyl Phosphate | 2.0 | |
| Tegocare 450 | Polyglyceryl-3 Methylglucose | | 3.0 |
| | Distearate | | |
| Lanette O | Cetearyl Alcohol | 3.0 | 3.0 |
| PARSOL ® 1789 | Butyl Methoxydibenzoyl Methane | 4.0 | |
| Parsol EHT | Ethylhexyl Triazone | 3.0 | |
| PARSOL ® 340 | Octocrylene | 8.0 | |
| HMS | Homosalate | 5.0 | |
| Uvinul A Plus | Diethylamino Hydroxybenzoyl | | 5.0 |
| | Hexyl Benzoate | | |
| Parsol MCX | Ethylhexyl Methoxycinnamate | | 8.0 |
| Parsol SLX | Polysilicone-15 | | 3.0 |
| Uvasrob HEB | Diethylhexyl Butamido Triazone | | 4.0 |
| Finsolv TN | C12-15 Alkyl Benzoate | 3.0 | 3.0 |
| Dub DI | Diisopropyl Sebacate | 2.0 | 2.0 |
| Myritol 318 | Caprylic/Capric Triglyceride | 10.0 | 8.0 |
| WATER DEM. | Aqua | Ad 100 | Ad 100 |
| Edeta BD | Disodium EDTA | 0.2 | 0.2 |
| Keltrol CG-T | Xanthan Gum | 0.2 | 0.2 |
| Glycerin 99.5% | Glycerin | 3.0 | 3.0 |
| 6-Sialyllactose | Sialyllactose | See | |
| 3-Sialyllactose | Sialyllactose | table 1b | See |
| | | | table 1c |
| Euxyl PE 9010 | Phenoxyethanol; | 1.0 | 1.0 |
| | Ethylhexylglycerin | | |

TABLE 1b
Results 6′Sialyllactose (Formulation 1)

| | Conc. HMO | SPF in | SPF |
|---|---|---|---|
| HMO | [wt.-%] | vitro | boosting |
| Reference | — | 21.8 | — |
| Sialyllactose (6′SL) | 1 | 27.1 | +24.6% |

TABLE 1c
Results 3′Sialyllactose (Formulation 2)

| | Conc. HMO | SPF in | SPF |
|---|---|---|---|
| HMO | [wt.-%] | vitro | boosting |
| Reference | — | 12.3 | — |
| Sialyllactose (3′SL) | 1 | 14.2 | +15.8% |

As shown in table 1b and 1c the HMO's 3SL and 6SL boost the in vitro SPF between 15% and 25%.

TABLE 2
Formulation 3

| | | Refer- | Inven- |
|---|---|---|---|
| | | ence | tion |
| trade name | INCI name | wt.-% | wt.-% |
| Lexfilm Sun Natural MB | Capryloyl | 1.5 | 1.5 |
| | Glycerin/Sebacic Acid | | |
| | Copolymer | | |
| Lanette ® O | Cetearyl Alcohol | 1.0 | 1.0 |
| TEGIN ® M Pellets MB | Glyceryl Stearate | 0.4 | 0.4 |
| AMPHISOL ® K | Potassium Cetyl | 0.5 | 0.5 |
| | Phosphate | | |
| Cetiol ® B | Dibutyl Adipate | 9.5 | 9.5 |
| DL-alpha-Tocopheryl | Tocopheryl Acetate | 0.5 | 0.5 |
| Acetate | | | |
| Uvinul A Plus | Diethylamino | 5.0 | 5.0 |
| | Hydroxybenzoyl Hexyl | | |
| | Benzoate | | |
| PARSOL ® EHT | Ethylhexyl Triazone | 1.5 | 1.5 |
| PARSOL ® Shield | Bis Ethylhexyloxyphenol | 2.0 | 2.0 |
| | Methoxyphenyl Triazine | | |
| PARSOL ® MCX | Ethylhexyl | 4.0 | 4.0 |
| | Methoxycinnamate | | |
| PARSOL SLX | Polysilicone-15 | 2.0 | 2.0 |
| Uvasaorb HEB | Diethylhexyl Butamido | 1.5 | 1.5 |
| | Triazone | | |
| X-Tend 226 | Phenethyl Benzoate | 5.5 | 5.5 |
| Dermosoft ® GMCY MB | Glyceryl Caprylate | 0.5 | 0.5 |
| TEGO CARE PBS 6 MB | Polyglyceryl-6 Stearate; | 4.0 | 4.0 |
| | Polyglyceryl-6 Behenate | | |
| HYA-ACT TM M | Sodium Hyaluronate | 0.1 | 0.1 |
| Phenethylalcohol Nat. | Phenethyl Alcohol | 1.0 | 1.0 |
| PARSOL ® MAX | Methylene Bis- | 4.0 | 4.0 |
| | Benzotriazolyl | | |
| | Tetramethylbutylphenol; | | |
| | Aqua; Decyl Glucoside; | | |
| | Propylene Glycol; | | |
| | Xanthan Gum | | |
| Water Dem | Aqua | Ad 100 | Ad 100 |
| 3′SL | Sialyllactose | | 1.00 |
| Rheocare ® XGN | XANTHAN GUM; | 0.4 | 0.4 |
| TILAMAR ® PDO with | PROPANEDIOL; | 2.0 | 2.0 |
| NØØVISTA ™ | | | |

| | | |
|---|---|---|
| SPF in vitro | 64.1 | 75.9 |
| SPF boosting | | 18% |

1. A composition comprising at least one UVA filter selected from the group of butyl methoxydibenzoyl methane and diethylamino hydroxybenzoyl hexyl benzoate and at least one sialylated human milk oligosaccharide.

2. The composition according to claim 1, wherein the at least one human milk oligosaccharide is present in an amount effective to increase SPF of the composition.

3. The composition according to claim 2, wherein the amount of the at least one sialylated human milk oligosaccharide is selected in the range from 0.2 to 5 wt.-%, based on the total weight of the composition.

4. The composition according to claim 1, wherein the at least one sialylated human milk oligosaccharide is 3′sialyllactose, 6′sialyllactose, or a saft thereof as well as salts thereof, preferably from 3′sialyllactose sodium salt and 6′sialyllactose sodium salt as well as mixtures thereof.

5. The composition according to claim 1, wherein the total amount of UVA filters selected from the group of butyl methoxydibenzoyl methane and diethylamino hydroxybenzoyl hexyl benzoate in the composition is selected in the range from 0.5 to 10 wt.-%, based on the total weight of the composition.

6. The composition according to claim 1, wherein the composition further comprises at least one organic UVB and/or broadband filter.

7. The composition according to claim 6, where in the at least one additional organic UVB and/or broadband filter is selected from the group consisting of homosalate, ethylhexyl salicylate, polysilicone-15, ethylhexyl methoxycinnamate, octocrylene, phenylbenzimidazole sulphonic acid, a salt of phenylbenzimidazole sulphonic acid, bis-ethylhexyloxyphenol methoxyphenyl triazine, ethylhexyl triazone, diethylhexyl butamido triazone, methylene bisbenzotriazolyl tetramethylbutylphenol, tris-biphenyl triazine, butyl methoxydibenzoylmethane, and diethylamino hydroxybenzoyl hexyl benzoate or a mixture thereof.

8. The composition according to claim 1, wherein the total amount of UV-filters in the composition is selected in the range from 1 to 50 wt.-%, based on the total weight of the composition.

9. The composition according to claim 1, wherein the composition comprises a carrier consisting of at least 30 wt. % of water.

10. The composition according to claim 1, wherein the composition is an emulsion.

11. The composition according to claim 10, wherein the O/W emulsion comprises an oily phase dispersed in an aqueous phase in the presence of an emulsifier selected from the group consisting of C8-C25 alkyl phosphate, polyglyceryl-6 stearate, polyglyceryl-6 behenate and polyglyceryl-3 methylglucose distearate, or a mixture thereof.

12. (canceled)

13. A method of increasing the sun protection factor (SPF) properties of a composition comprising at least one UVA filter selected from the group of butyl methoxydibenzoyl methane and diethylamino hydroxybenzoyl hexyl benzoate, said method comprising adding at least one sialylated human milk oligosaccharide to the composition in an amount effective to increase the SPF properties of the composition.

14. A method of preparing a composition comprising at least one UVA filter selected from the group of butyl methoxydibenzoyl methane and diethylamino hydroxybenzoyl hexyl benzoate and at least one sialylated human milk oligosaccharide to the composition during preparation of the composition.

15. (canceled)

16. The composition according to claim 1, wherein the at least one sialylated human milk oligosaccharide is 3′sialyllactose sodium salt, 6′sialyllactose sodium salt, or a mixture thereof.

17. The composition according to claim 3, wherein the amount of the at least one sialylated human milk oligosaccharide is selected in the range from 0.5 to 4 wt.-%, based on the total weight of the composition.

18. The composition according to claim 5, wherein the total amount of UVA filters is selected in the range from 2 to 6 wt.-%, based on the total weight of the composition.

19. The composition according to claim 7, wherein in the at least one additional organic UVB and/or broadband filter is selected from the group consisting of ethylhexyltriazone, octocrylene, homosalate, ethylhexyl methoxycinnamate and polysilicone-15; or a mixture thereof.

20. The composition according to claim 8, wherein the total amount of UV-filters in the composition is selected in the range from 5 to 30 wt.-%, based on the total weight of the composition.

21. The composition according to claim 9, wherein the composition comprises a carrier consisting of at least 45 wt.-% of water.

22. The composition according to claim 10, wherein the emulsion is an O/W emulsion.
